(19) United States
(12) Patent Application Publication
(10) Pub. No.: US 2023/0029222 A1
(43) Pub. Date: Jan. 26, 2023

(54) WEARABLE DEVICE AND METHOD FOR PROCESSING ACCELERATION DATA

(71) Applicant: POSTECH Research and Business Development Foundation, Pohang-si (KR)

(72) Inventors: Yoonyoung CHUNG, Pohang-si (KR); Young-Seok KIM, Seongnam-si (KR); Inyeol YUN, Pohang-si (KR)

(21) Appl. No.: 17/481,220

(22) Filed: Sep. 21, 2021

(30) Foreign Application Priority Data
Jul. 13, 2021 (KR) 10-2021-0091282

(51) Int. Cl. A61B 5/00; A61B 5/11; G06N 20/20; G06N 20/10

(52) U.S. Cl. CPC A61B 5/4094; A61B 5/746; A61B 5/6813; A61B 5/1117; G06N 20/20; G06N 20/10; A61B 5/7264; A61B 2562/0219

(57) ABSTRACT

According to an embodiment, a method of processing acceleration information by a wearable device is provided, the method including: acquiring the acceleration information indicating acceleration according to movement of the wearable device; acquiring a plurality of acceleration characteristics including a plurality of characteristics of a power spectrum of the acceleration and a range of the acceleration on the basis of the acceleration information; determining characteristic values according to two characteristics determined according to a type of a disease to be monitored among the plurality of acceleration characteristics; and providing an alarm signal in response to the characteristic value falling within an abnormality range.

### TECHNICAL FIELD

The present disclosure relates to a technology for processing acceleration information, and more specifically, to a technology for providing information about whether a human body has an abnormal disease by detecting vibrations occurring from the human body through a wearable sensor.

### BACKGROUND ART

In general, when measuring and analyzing signals generated from a human body using a wearable sensor, machine learning algorithms are used to acquire high accuracy. However, since the machine learning algorithms require high computational power, the conventional wearable sensor needs to transmit all measured signals to a data center.

Since such wireless communication occupies a large portion of the power consumption of the wearable sensor, the machine learning analysis technique in the data center through wireless transmission of all pieces of raw data according to the related art is very inefficient in terms of power consumption.

Accordingly, research is being conducted to process the abnormality discrimination of signals measured by a wearable sensor directly inside the wearable sensor, but since wearable sensors have a relatively low computational power, analyzing signals only with a simple signal processing method exhibits a low accuracy of signal analysis.

Among the related arts, a linear discriminant analysis (LDA) technology is a representative signal processing method that is included in a wearable sensor to discriminate a vibration signal and operates to project pieces of data on a straight line to classify the data on the basis of a mean and a variance between the pieces of data desired to classify, and due to a characteristic of a low computation, embedding in a wearable sensor is easy but the accuracy is greatly lowered when analyzing complex data.

Accordingly, there is a growing demand for analysis technology that may solve the above limitations and enhance the efficiency in terms of power consumption and accuracy.

### DISCLOSURE

### Technical Problem to be Solved

An embodiment of the present disclosure provides an improved technology in terms of power consumption and accuracy while processing abnormality discrimination of a signal measured by a wearable sensor directly inside the wearable sensor.

The technical objectives of the present disclosure are not limited to the above, and other objectives may become apparent to those of ordinary skill in the art based on the following description.

### Technical Problem to be Solved

According to the first aspect of the present disclosure, there is provided a method of processing acceleration information by a wearable device, the method including: acquiring the acceleration information indicating acceleration according to movement of the wearable device; acquiring a plurality of acceleration characteristics including a plurality of characteristics of a power spectrum of the acceleration and a range of the acceleration on the basis of the acceleration information; determining characteristic values according to two characteristics determined according to a type of a disease to be monitored among the plurality of acceleration characteristics; and providing an alarm signal in response to the characteristic value falling within an abnormality range.

The providing of the alarm signal may include, in response to the range of the acceleration exceeding a preset value, transmitting the alarm signal to an external device.

The plurality of acceleration characteristics may include at least one of a first characteristic indicating a range of the acceleration in a time domain, a second characteristic indicating a root mean square of the acceleration in the time domain, and a third characteristic indicating an average of the acceleration in the time domain.

The plurality of acceleration characteristics may further include at least one of a fourth characteristic indicating a mean frequency for the power spectrum of the acceleration, a fifth characteristic indicating a frequency including a preset first proportion of a total power for the power spectrum of the acceleration, a sixth characteristic indicating a frequency including a second proportion of the total power, a seventh characteristic indicating the total power, and an eighth characteristic indicating a power ratio at a frequency less than a preset motion noise frequency to the total power.

In response to the type of the disease being a first disease indicating a cough, the two characteristics corresponding to the first disease may include the fifth characteristic and the eighth characteristic, in response to the type of the disease being a second disease indicating an epileptic seizure, the two characteristics corresponding to the second disease may include the fourth characteristic and the eighth characteristic, and in response to the type of the disease being a third disease indicating a fall, the two characteristics corresponding to the third disease may include the first characteristic and the fourth characteristic.

The providing of the alarm signal in response to the characteristic value being included in the abnormality range may include determining the abnormality range in one or more ranges using a straight line defined by one or more preset linear equations or a curve defined by a polynomial equation on a two-dimensional (2D) plane.

The determining of the characteristic values according to the two characteristics may include determining the two characteristics and the abnormality range on the basis of learning data that is previously generated, wherein the learning data may be acquired on the basis of at least one of a support vector classifier (SVC), a decision tree, Naïve Bayes, a random forest, and a neural network.

The providing of the alarm signal in response to the characteristic value falling within the abnormality range may include determining whether the characteristic value falls within the abnormality range using a hyperplane for the plurality of acceleration characteristics acquired through a support vector machine.

According to the second aspect of the present disclosure, there is a wearable device for processing acceleration information, the wearable device including: an acceleration sensor configured to acquire the acceleration information which indicates acceleration according to movement of the wearable device; a processor configured to acquire a plurality of acceleration characteristics including a plurality of characteristics of a power spectrum of the acceleration and a range of the acceleration based on the acceleration information and determine characteristic values according to two characteristics determined according to a type of a disease to be monitored among the plurality of acceleration characteristics; and a transmitter configured to provide an alarm signal in response to the characteristic value falling within an abnormality range.

The transmitter may be configured to, in response to the range of the acceleration exceeding a preset value, transmit the alarm signal to an external device.

The plurality of acceleration characteristics may include at least one of a first characteristic indicating a range of the acceleration in a time domain, a second characteristic indicating a root mean square of the acceleration in the time domain, and a third characteristic indicating an average of the acceleration in the time domain.

The plurality of acceleration characteristics may further include at least one of a fourth characteristic indicating a mean frequency for the power spectrum of the acceleration, a fifth characteristic indicating a frequency including a preset first proportion of a total power for the power spectrum of the acceleration, a sixth characteristic indicating a frequency including a second proportion of the total power, a seventh characteristic indicating the total power, and an eighth characteristic indicating a power ratio at a frequency less than a preset motion noise frequency to the total power.

In response to the type of the disease being a first disease indicating a cough, the two characteristics corresponding to the first disease may include the fifth characteristic and the eighth characteristic, in response to the type of the disease being a second disease indicating an epileptic seizure, the two characteristics corresponding to the second disease may include the fourth characteristic and the eighth characteristic, and in response to the type of the disease being a third disease indicating a fall, the two characteristics corresponding to the third disease may include the first characteristic and the fourth characteristic.

The processor may be configured to determine the abnormality range in one or more using a straight line defined by one or more preset linear equations or a curve defined by a polynomial equation on a two-dimensional (2D) plane.

The processor may be configured to determine the two characteristics and the abnormality range on the basis of learning data that is previously generated, wherein the learning data may be acquired on the basis of at least one of a support vector classifier (SVC), a decision tree, Naïve Bayes, a random forest, and a neural network.

According to the third aspect of the present disclosure, there is provided a recording medium on which a program for executing the method according to the first aspect is recorded.

According to the fourth aspect of the present disclosure, there is provided a computer program stored in a recording medium to implement the method according to the first aspect.

### Effects of the Invention

According to an embodiment of the present disclosure, since information can be directly processed with little computation without going through an external device by selecting two optimal characteristics for abnormality discrimination according to a disease to be monitored, power consumption used for wireless communication can be significantly improved.

In addition, characteristic values and abnormality ranges of two characteristics selected for each disease are analyzed on a two-dimensional (2D) plane on the basis of support vector machine (SVM), thereby providing efficient results in terms of both resource consumption and accuracy.

In addition, power is controlled to be supplied to main components when acceleration with a significant size is detected so that power consumption can be improved.

It should be understood that the effects of the present disclosure are not limited to the above effects and include all effects that can be deduced from the detailed description of the present disclosure or the configuration of the present disclosure described in the claims.

### DESCRIPTION OF DRAWINGS

FIG. 1 is a block diagram illustrating a configuration of a wearable healthcare sensor system (1000) according to an embodiment.

FIG. 2 is a block diagram illustrating an example of a configuration of a wearable device (100) according to an embodiment, and FIG. 3 is a flowchart showing a method of processing acceleration information by a wearable device (100) according to an embodiment.

FIGS. 4A through 4E are illustrations of an operation of determining one or more abnormality ranges on a two-dimensional plane by a wearable device (100) according to an embodiment.

FIGS. 5 to 7 are diagrams for describing operations of determining an existence of an abnormality using one or more abnormality ranges determined on a two-dimensional plane for first to third diseases by a wearable device (100) according to an embodiment.

FIG. 8A shows a block diagram illustrating a configuration of a switch circuit included in a wearable device (100) according to an embodiment, and FIG. 8B shows a diagram illustrating a comparison result of power consumption according to the presence or absence of the switch circuit.

FIG. 9 is a block diagram illustrating a configuration of a wearable healthcare sensor system (1000) according to another embodiment.

### MODES OF THE INVENTION

Although terms used herein are selected from among general terms that are currently and widely used in consideration of functions in the exemplary embodiments, these may be changed according to intentions or customs of those skilled in the art or the advent of new technology. However, when a specified term is defined and used in an arbitrary sense, a meaning of the term will be described in the specification in detail. Accordingly, the terms used herein are not to be defined as simple names of the components but should be defined based on the actual meaning of the terms and the whole context throughout the present specification.

Throughout the specification, the term “comprises” or “includes” and/or “comprising” or “including” means that one or more other components may further be not excluded unless context dictates otherwise. In the specification, the term “part” or “module” refers to a unit for processing at least one function or operation that may be implemented in hardware, software, or a combination thereof.

Throughout the specification, an expression “providing” may be interpreted as including a process in which an object acquires specific information or directly or indirectly transmits or receives specific information to or from a specific object, and comprehensively including the performance of relevant operations required in the process.

Although embodiments of the present disclosure will be described in detail with reference to the accompanying drawings in order to enable those skilled in the art to easily practice the disclosure, the present disclosure may be embodied in many different forms and should not be construed as being limited to the embodiments set forth herein.

Hereinafter, embodiments of the present disclosure will be described in detail with reference to the drawings.

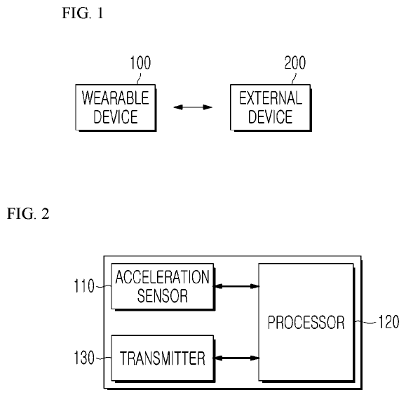

FIG. 1 is a block diagram illustrating a configuration of a wearable healthcare sensor system 1000 according to an embodiment.

Referring to FIG. 1, a wearable healthcare sensor system 1000 may include one or more wearable devices 100 and an external device 200.

The wearable device 100 corresponds to a computing device capable of processing acceleration information. In one embodiment, the wearable device 100 may be implemented as a health care device in a form detachable from a human body of a user to detect and process acceleration information due to movement of the human body, but the present disclosure is not limited thereto, and in another embodiment, the wearable device may be implemented as a vibration measurement device that is attached to various mechanical devices that generate vibration (e.g., vibration measurement of large structures, abnormal vibration monitoring and equipment diagnosis of various plants, vibration measurement for vibration pollution investigation, vibration mode analysis of sporting goods, etc.) to detect and process acceleration information of the mechanical device.

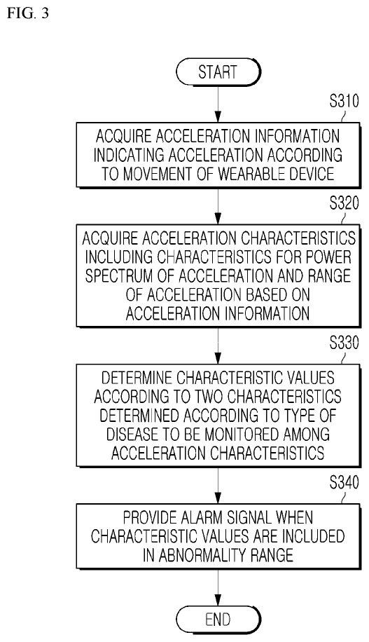

The wearable device 100 may determine whether abnormality exists for one or more diseases to be monitored on the basis of a plurality of acceleration characteristics acquired from acceleration information, and upon determining an abnormality exists, may provide an alarm signal corresponding thereto. Details thereof will be described below with reference to FIGS. 2 to 9.

The external device 200 corresponds to a computing device capable of receiving information provided from the wearable device 100 and storing the received information. In one embodiment, the external device 200 may be implemented as a computer, such as a desktop personal computer (PC), a tablet PC, and a laptop PC, and in another embodiment, may be implemented as a data server capable of collecting data and managing data on the basis of a cloud (e.g., a public cloud, a private cloud, etc.). In an embodiment, the external device 200 may be connected to one or more wearable devices 100 through a wireless network. Here, the wireless network may include various wireless communication networks, such as a wireless local area network (LAN), a low powered wide area (LPWA) network, etc.

FIG. 2 is a block diagram illustrating an example of a configuration of a wearable device 100 according to an embodiment, and FIG. 3 is a flowchart showing a method of processing acceleration information by a wearable device 100 according to an embodiment.

Referring to FIGS. 2 and 3, the wearable device 100 may include an acceleration sensor 110, a processor 120, and a transmitter 130.

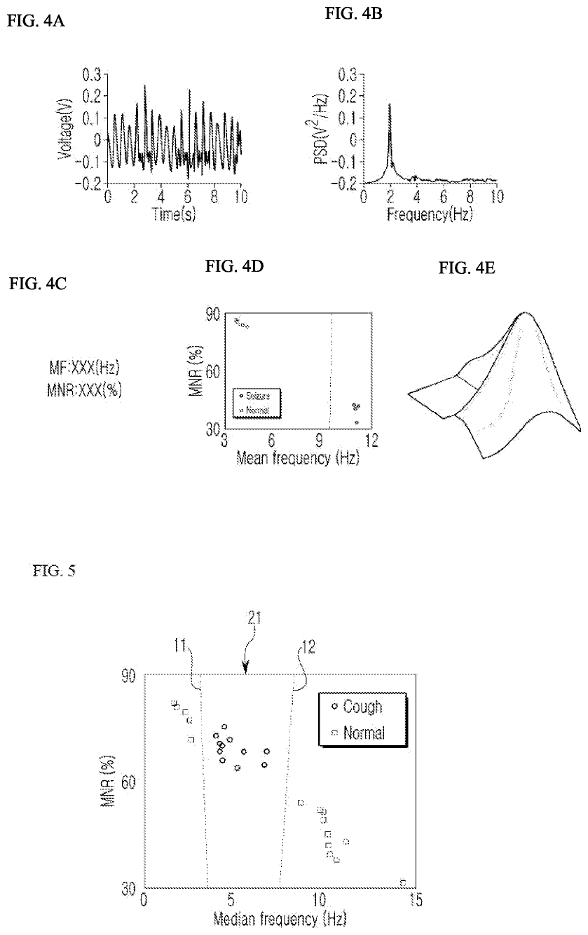

In operation S310, the acceleration sensor 110 may acquire acceleration information indicating acceleration according to movement of the wearable device 100. In an embodiment, the acceleration sensor 110 may acquire acceleration information including a change in the speed per unit time of the wearable device 100 and, for example, as illustrated in FIG. 4A, may sense acceleration information including a voltage level of an acceleration signal over time.

In an embodiment, the acceleration sensor 110 may acquire acceleration information including output information of an acceleration signal acquired in a time domain on the basis of three axes (e.g., x, y, and z axes) and, to this end, may be implemented as a 3-axis accelerometer that detects acceleration in three-axis directions. For example, the acceleration sensor 110 may determine the acceleration by integrating acceleration measurement results in each of the three axis directions according to Equation 1 below.

V_{acc}=\sqrt{Vₓ²+V_{y}²+V_{z}²} [Equation 1]

(here, Vₓ, V_{y}, and V_{z} represent outputs (e.g., voltage levels) of acceleration signals measured on the x-axis, y-axis, and z-axis, respectively, and V_{acc} represents an output of the acceleration (e.g., a voltage level) integrating the outputs of the acceleration signals on the respective axes.

In operation S320, the processor 120 may acquire a plurality of acceleration characteristics on the basis of the acquired acceleration information. Here, the plurality of acceleration characteristics includes a plurality of characteristics for a power spectrum of the acceleration and a range of the acceleration.

More specifically, the processor 120 may determine some of the plurality of acceleration characteristics through analysis of the acceleration in the time domain included in the acceleration information, convert the acceleration in the time domain into a frequency domain, and determine the remaining of the plurality of acceleration characteristics through analysis of a power spectrum of the acceleration. In an embodiment, the plurality of acceleration characteristics may be determined on the basis of Table 1 below.

TABLE 1

| Characteristic | Domain | Characteristic Definition [unit] |
|---|---|---|
| first | time | range of acceleration signals |
| characteristic | | [m/s²] |
| second | time | root mean square (RMS) of |
| characteristic | | accelerations signals [m/s²] |
| third | time | average of acceleration signals |
| characteristic | | [m/s²] |
| fourth | frequency | mean frequency of power |
| characteristic | | spectrum of acceleration [Hz] |
| fifth | frequency | frequency containing 50% of |
| characteristic | | the total power for power |
| | | spectrum of acceleration [Hz] |
| sixth | frequency | frequency containing 95% of |
| characteristic | | the total power for power |
| | | spectrum of acceleration [Hz] |
| seventh | frequency | total power of power spectrum |
| characteristic | | of accelerations [m²/s⁴ · Hz] |
| eighth | frequency | power ratio at frequency less |
| characteristic | | than 10 Hz to total power |

The processor 120 may acquire information about the power spectrum of the acceleration through frequency conversion of the acceleration to acquire a plurality of acceleration characteristics. In an embodiment, as illustrated in FIG. 4B, the processor 120 may determine the power spectral density (PSD) of the acceleration in the frequency domain using a discrete Fourier transform (DFT), and for example, may convert an acceleration signal in the time domain into the frequency domain by performing a Fast Fourier Transform (FFT) for calculating an approximation value of a function on the basis of Equation 2 below.

$$P [ f ] = {\sum }_{n = 0}^{N - 1} x [ n ] \cdot {e}^{- jk \frac{2 π}{N} n} \qquad [ Equation 2 ]$$

(here, P[f] represents the power spectral density of V_{acc}, and N represents a configurable length of measurement data)

The processor 120 may determine a plurality of acceleration characteristics for the movement of the wearable device 100 on the basis of the information about the acceleration in the time domain and the information about the power spectrum of the acceleration.

In an embodiment, the plurality of acceleration characteristics may include a first characteristic indicating a range of acceleration in the time domain. For example, the processor 120 may extract the range of the acceleration signal from the acceleration in the time domain according to Equation 3 below and determine the range of the acceleration signal as the first characteristic value.

Range=max(V_{acc})−min(V_{acc}) [Equation 3]

(herein, max(V_{acc}) represents an operation of obtaining the maximum voltage value of V_{acc} within a specified time range in the time domain, and min(V_{acc}) represents an operation of obtaining the minimum voltage value of V_{acc} within the time range.

In an embodiment, the plurality of acceleration characteristics may include a second characteristic indicating a root mean square (RMS) of acceleration in the time domain. For example, the processor 120 may extract an RMS of the entire acceleration signal from the acceleration in the time domain according to Equation 4 below and determine the RMS as the second characteristic value.

$$RMS = \sqrt{\frac{1}{N} {\sum }_{n = 1}^{N} {\vert {V}_{acc , n} \vert}^{2}} \qquad [ Equation 4 ]$$

In an embodiment, the plurality of acceleration characteristics may include a third characteristic indicating a mean (a mean acceleration: MA) of the acceleration in the time domain. For example, the processor 120 may extract the mean acceleration from the acceleration in the time domain according to Equation 5 below and determine the mean acceleration as the third characteristic value.

$$MA = \frac{{\sum }_{n = 1}^{N} {V}_{acc , n}}{N} \qquad [ Equation 5 ]$$

In an embodiment, the plurality of acceleration characteristics may include a fourth characteristic indicating a mean frequency (MF) for the power spectrum of the acceleration. For example, the processor 120 may extract the MF from the power spectrum of the acceleration in the frequency domain according to Equation 6 below and determine the MF as the fourth characteristic value.

$$MF = \frac{{\sum }_{0}^{fs / 2} f \cdot P ( f ) df}{{\sum }_{0}^{fs / 2} P ( f ) df} \qquad [ Equation 6 ]$$

(here, P(f) represents the power spectral density of V_{acc}, N represents the length of measurement data, and fₛ represents a configurable measurement sampling frequency).

In an embodiment, the plurality of acceleration characteristics may include a fifth characteristic indicating a frequency (e.g., F50) including a preset first proportion (e.g., 50%) of the total power (TP) of the power spectrum of the acceleration. For example, the processor 120 may extract a median frequency including 50% of the entire signal from the power spectrum for the acceleration in the frequency domain according to Equation 7 below and determine the median frequency as the fifth characteristic value.

$${\sum }_{0}^{F 50} P ( f ) df = \frac{1}{2} {\sum }_{0}^{fs / 2} P ( f ) df \qquad [ Equation 7 ]$$

In an embodiment, the plurality of acceleration characteristics may include a sixth characteristic indicating a frequency (e.g., F95) including a second proportion (e.g., 95%) of the TP of the power spectrum of the acceleration. For example, the processor 120 may extract a frequency including 95% of the entire signal from the power spectrum for the acceleration in the frequency domain according to Equation 8 below and determine the frequency as the sixth characteristic value.

$${\sum }_{0}^{F 95} P ( f ) df = \frac{19}{20} {\sum }_{0}^{fs / 2} P ( f ) df \qquad [ Equation 8 ]$$

In an embodiment, the plurality of acceleration characteristics may include a seventh characteristic indicating a TP of the power spectrum of the acceleration. For example, the processor 120 may extract the total signal power from the power spectrum for the acceleration in the frequency domain according to Equation 9 below and determine the total signal power as the seventh characteristic value.

$$TP = {\sum }_{0}^{fs / 2} P ( f ) df \qquad [ Equation 9 ]$$

In an embodiment, the plurality of acceleration characteristics may include an eighth characteristic indicating a power ratio (a motion noise ratio: MRN) at a frequency less than a preset motion noise frequency, e.g., 10 Hz, to the TP of the power spectrum of the acceleration, (for example, a ratio of noise due to a motion in the total power spectral density). For example, the processor 120 may extract a ratio of noise due to a motion from the power spectrum for the acceleration in the frequency domain according to Equation 10 below and determine the noise ratio as the eighth characteristic value.

$$MNR = \frac{{\sum }_{0}^{10} P ( f ) df}{TP} \qquad [ Equation 10 ]$$

In an embodiment, the plurality of acceleration characteristics may include two or more of the first to eighth characteristics, but the disclosure is not limited thereto. In another embodiment, the plurality of acceleration characteristics may include various characteristics that may be extracted from the time domain and the frequency domain of the signal, for example, a power ratio of a specific frequency band to the entire signal.

In an embodiment, the types of the plurality of acceleration characteristics may be determined on the basis of a learning algorithm before operation S310. For example, as machine learning-based learning is performed by the external device 200, information about the type of an acceleration characteristic to be extracted from the acceleration information may be determined in advance, and the processor 120 may perform operation S320 using the information about the type of the acceleration characteristic that is received from the external device 200 or stored in advance.

In operation S330, the processor 120 may determine characteristic values according to two characteristics determined according to the type of disease to be monitored among the acquired plurality of acceleration characteristics. In an embodiment, the processor 120 may determine two characteristics corresponding to the type of disease to be monitored from among the plurality of acceleration characteristics. For example, the processor 120 may determine one or more types of disease to be monitored by fetching a user set value stored in advance for a monitoring target from a memory or by receiving a user input thereof and may determine two characteristics corresponding to each of the determined types of disease on the basis of Table 2 below.

TABLE 2

| Types of disease | Characteristics |
|---|---|
| cough | fifth characteristic and eighth |
| | characteristic |
| epileptic seizure | fourth characteristic and eighth |
| | characteristic |
| fall | first characteristic and fourth |
| | characteristic |

As shown in Table 2, when the type of disease to be monitored is the first disease indicating a cough, the two characteristics corresponding to the first disease may be the fifth characteristic and the eighth characteristic. For example, the processor 120, based on a previously stored corresponding relationship between types of disease and two characteristics, may only extract the fifth characteristic value (e.g., F50) and the eighth characteristic value (MNR) for monitoring a cough among the first to eighth characteristics acquired from the acceleration information and may use the extracted fifth and eight characteristics as information for cough monitoring in a subsequent operation.

In an embodiment, when the type of disease to be monitored is a second disease indicating an epileptic seizure, the two characteristics corresponding to the second disease may be the fourth characteristic and the eighth characteristic. For example, as illustrated in FIG. 4C, the processor 120, based on the previously stored corresponding relationship between types of disease and two characteristics, may only extract the fourth characteristic value (e.g., MF) and the eighth characteristic value (MNR) for monitoring an epileptic seizure among the first to eighth characteristics acquired from the acceleration information.

In an embodiment, when the type of disease to be monitored is a third disease indicating a fall, the two characteristics corresponding to the third disease may be the first characteristic and the fourth characteristic. For example, the processor 120, based on the previously stored corresponding relationship between types of disease and two characteristics, may only extract the first characteristic value (e.g., range) and the fourth characteristic value (e.g., MF) for monitoring a fall among the first to eighth characteristics acquired from the acceleration information.

As described above, the types of disease may include at least one of the first disease indicating a cough, the second disease indicating an epileptic seizure, and a third disease indicating a fall, but the present disclosure is not limited thereto, and in another embodiment, the types of disease may be broadly interpreted as a meaning encompassing various types of illness or diseases related to the human body, and various types of vibration measurement related to machines or objects.

In an embodiment, the processor 120 may first extract first to eighth characteristic values from acceleration information and then fetch two characteristic values according to the type of disease to be monitored among the first to eighth characteristic values. In another embodiment, the processor 120 may selectively extract characteristic values for only two characteristics according to the type of disease to be monitored from the acceleration information.

In an embodiment, the processor 120 may determine two characteristics according to the type of disease to be monitored on the basis of previously generated first learning data. For example, the processor 120 may determine two characteristics according to the type of disease using the first learning data that may be received from the external device 200 before operation S310 or embedded in the wearable device 100.

In an embodiment, the external device 200 may generate the first learning data for determining two characteristics according to the type of disease using a first algorithm before operation S310. For example, the external device 200 may analyze the most sensitive characteristic among the first to eighth characteristics with respect to each type of diseases to be monitored on the basis of a support vector classifier (SVC) algorithm (e.g., a radial basis function (RBF) function), which is configured to obtain the best classifier based on the distance between each piece of data for a data set to be distinguished, and previously measured experimental data, may generate first learning data including a result of determining the best two characteristics for each type of disease according to the analysis result and may update the first learning data through continuous learning and provide the wearable device 100 with the updated first learning data.

In operation S340, the transmitter 130 may provide an alarm signal when the characteristic values according to the two determined characteristics are included in abnormality ranges. In an embodiment, when the characteristic value of the first characteristic indicating the range of acceleration exceeds a preset value, the transmitter 130 may transmit an alarm signal to the external device 200. For example, on the basis of a preset abnormality range for each of the plurality of characteristics, the processor 120 may compare the characteristic values according to the two characteristics with abnormality ranges corresponding thereto, and when all of the characteristic values are included within the corresponding abnormality ranges as a result of the comparison, determine that an abnormality has occurred for the corresponding disease, and may transmit an alarm signal including a message notifying a user of at least one among a characteristic type, a characteristic value, an abnormality range, and an abnormality occurrence to the external device 200.

In an embodiment, the processor 120 may determine one or more abnormality ranges using a straight line defined by one or more preset linear equations or a curve defined by a polynomial equation on a two-dimensional (2D) plane. For example, as illustrated in FIG. 4D, the processor 120 may acquire the characteristic values of two characteristics (e.g., MF and MNR) determined corresponding to a disease (e.g., an epileptic seizure) to be monitored as coordinate information (e.g., (m, n)) represented on a 2D plane, and according to a linear function (e.g., y=ax+b) and an abnormality condition (e.g., located below the straight line y=ax+b on the 2D plane), which are preset corresponding to the disease (e.g., an epileptic seizure), may determine one or more abnormality ranges (e.g., a set of (m, n) located below the line y=ax+b on the 2D plane). In another embodiment, the processor 120 may determine the abnormal range using a curve defined by a preset quadratic equation on a 2D plane.

In an embodiment, the processor 120 may determine whether a characteristic value determined using a hyperplane for a plurality of acceleration characteristics acquired through an SVM is included in an abnormal range. Here, the hyperplane may refer to a reference plane optimized for dividing sets of signals to be distinguished when measured signals are displayed on an n-dimensional graph, and in an embodiment, the hyperplane may be understood as a concept including the straight line defined by the linear equation and the curve defined by the quadratic equation as described above. For example, as a hyperplane for each signal discrimination is acquired by the external device 200 using an SVM before operation S310, a hyperplane-based discriminator is stored in the wearable device 100, and as illustrated in FIG. 4E, the processor 120, in response to acceleration information being measured, may check whether two characteristics extracted for each disease are included within an abnormality range distinguished according to the hyperplane using a previously stored SVM-based discriminator so that whether the disease is abnormal may be rapidly determined with low power consumption.

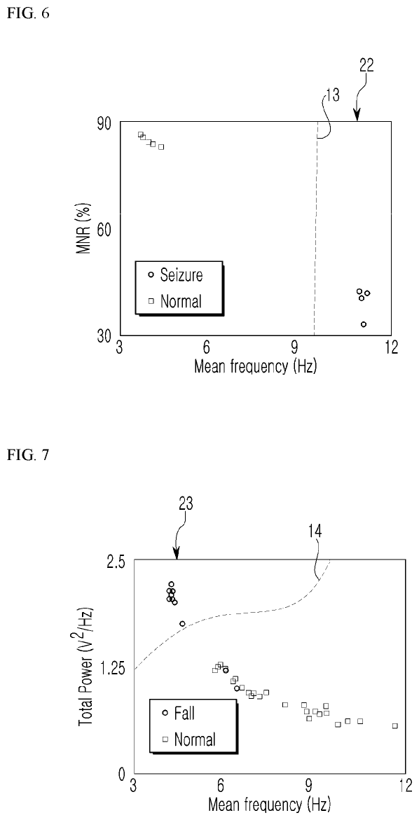

Hereinafter, the operation will be described in more detail with further reference to FIGS. 5 to 7.

FIGS. 5 to 7 are diagrams for describing operations of determining an existence of an abnormality using one or more abnormality ranges determined on a two-dimensional plane for first to third diseases by a wearable device 100 according to an embodiment.

Referring to FIG. 5, when two characteristics for discriminating a cough include a median frequency (F50) (e.g., x domain) and MNR (e.g., y domain), the processor 120 may analyze a first straight line 11 according to a first function (e.g., y=ax+b, wherein a and b are real numbers) and a second straight line 12 according to a second function (e.g., y=cx+d, wherein c and d are real numbers), which are previously stored as a hyperplane for a cough, on the x-y plane, and may determine a region between the first straight line 11 and the second straight line 12 as a first abnormality range 21 according to a preset abnormality condition. In addition, in a case in which a value of F50 and a value of MNR extracted from the acceleration information are m₁ and n₁, when the values (m₁, n₁) are included in the first abnormality range 21 on the x-y plane, the processor 120 may determine that a cough has occurred. For example, in a case in which the first abnormality range 21 is a region between y=ax+b and y=cx+d, when the measured values (m₁, n₁) satisfy a condition “c m₁+d<n₁<am₁+b,” a cough may be determined.

Referring to FIG. 6, when two characteristics for discriminating an epileptic seizure include MF (e.g., x domain) and MNR (e.g., y domain), the processor 120 may analyze a third straight line 13 according to a third function (e.g., y=ex+f, wherein and f are real numbers), which is previously stored as a hyperplane for an epileptic seizure, on the x-y plane, and may determine a region on the right side of the third straight line 13 according to a preset abnormality condition as a second abnormality range 22. In addition, in a case in which a value of MF and a value of MNR extracted from the acceleration information are m₂ and n₂, when the values (m₂, n₂) are included in the second abnormality range 22 on the x-y plane, the processor 120 may determine that an epileptic seizure has occurred. For example, in a case in which the second abnormality range 22 is located below y=ex+f, when the measured values (m₂, n₂) satisfy a condition “n₂<em₂+f,” an epileptic seizure may be determined.

Referring to FIG. 7, when two characteristics for discriminating a fall include MF (e.g., x domain) and TP (e.g., y domain), the processor 120 may analyze a fourth curve 14 according to a fourth function (e.g., y=gx²+hx+I, wherein g, h, and i are real numbers), which is previously stored as a hyperplane for a fall, on the x-y plane, and may determine a region located above the curve 14 as a third abnormality range 23 according to a preset abnormality condition. In addition, in a case in which a value of MF and a value of TP extracted from the acceleration information are m₃ and n₃, when the values (m₃, n₃) are included in the third abnormality range 23 on the x-y plane, the processor 120 may determine that a fall has occurred. For example, in a case in which the third abnormality range 23 is located above y=gx²+hx+I, when the measured values (m₃, n₃) satisfy a condition “y=gm₃²+hm₃+I<n₃,” a fall may be determined as having occurred.

The processor 120 may acquire an abnormality range for a disease to be monitored or information about one or more linear equations used to determine the abnormality range before operation S310. In an embodiment, the processor 120 may determine abnormality ranges for two characteristics according to the type of disease to be monitored on the basis of second learning data that is previously generated. For example, the processor 120 may determine abnormality ranges for two characteristics according to the type of disease using the second learning data that may be received from the external device 200 before operation S310 or embedded in the wearable device 100.

In an embodiment, the external device 200 may generate the second learning data for determining abnormality ranges of two characteristics determined according to the type of disease using a second algorithm before operation S310. For example, the external device 200 may acquire second learning data for determining ranges of two features selected for each type of disease, in which a measured signal is an abnormal signal, on the basis of a machine learning algorithm and experimental data measured in advance, update the second learning data through continuous learning, and provide the wearable device 100 with the second learning data.

In an embodiment, the first learning data and the second learning data may be acquired on the basis of at least one of SVC, a decision tree, naïve Bayes, a random forest, and a neural network, but the present disclosure is not limited thereto, and various other machine learning algorithms may be applied.

As such, the wearable device 100 may select eight characteristics from the acceleration information to determine the presence or absence of an abnormality for various diseases, and using a result of analyzing a characteristic that is the most sensitive to a type of disease to be detected among the selected eight characteristics according to a prior learning algorithm, determine whether there is an abnormality for the disease.

Accordingly, since the wearable device 100 may directly process information with only a small amount of computation without going through the external device 200 by selecting two optimal characteristics for discriminating an abnormality of a disease to be monitored, power consumption used for communication can be remarkably improved.

In addition, the wearable device 100 can improve the accuracy of abnormality discrimination by analyzing information with respect to the abnormality range on a 2D plane based on an SVM.

In an embodiment, the processor 120 may determine the accuracy on the basis of the degree to which the two determined characteristic values deviate from a normal range. For example, when monitoring an epileptic seizure, the processor 120 may calculate a perpendicular distance between coordinates (m, n) according to two characteristic values of MF and MNR extracted from acceleration information and the third straight line 13 corresponding to the hyperplane and may calculate the accuracy in proportion to the perpendicular distance. For example, in response to being close to the boundary of the straight line corresponding to the hyperplane, the processor 120 may calculate the accuracy to be relatively low. In addition, in an embodiment, the processor 120 may determine the accuracies on the basis of different weights that are given according to each type of disease and, for example, may calculate the accuracies by reflecting the weights in which a larger weight is assigned in the order of a cough, an epileptic seizure, and a fall.

In an embodiment, when the accuracy is less than a preset value, the processor 120 may use a characteristic value of an additional characteristic preset for the type of the disease to determine whether the disease is abnormal. For example, when the characteristic values of MF and TP extracted from acceleration information for a fall are calculated with a low accuracy due to being close to the boundary of a straight line corresponding to the hyperplane on a 2D plane, the processor 120 may extract a range value, which is an additional characteristic additionally set for a fall, and determine whether an abnormality for a fall exists by identifying whether the range value is included in an abnormality range. In an embodiment, the abnormality range of the additional characteristic may be determined in a different way from those of the two characteristics. For example, a range of a one-dimensional value preset for the additional characteristic may be simply applied unlike the two characteristics, and the additional characteristic may also be determined through prior learning.

In an embodiment, the processor 120 may perform a series of operations for processing acceleration information and may be electrically connected to the acceleration sensor 110, the transmitter 130, and other components to control a data flow therebetween. In an embodiment, the processor 120 may be implemented to include a central processor unit (CPU) that controls overall operations of the wearable device 100 and may be implemented, for example, in the form of a microcontroller unit (MCU) including a CPU core, a memory, and programmable input and output. In addition, in an embodiment, the transmitter 130 may be implemented to include a communication module for transmitting and receiving information to and from the external device 200 or another device (e.g., a server, a terminal, etc.) through a network.

Meanwhile, in addition to the components shown in FIG. 2, other general-purpose components may be further included in the wearable device 100. For example, the wearable device 100 may further include a storage module (e.g., a memory, a cloud, etc.) for storing information described throughout the specification, a user interface for receiving user input, a display for displaying monitoring results, and the like.

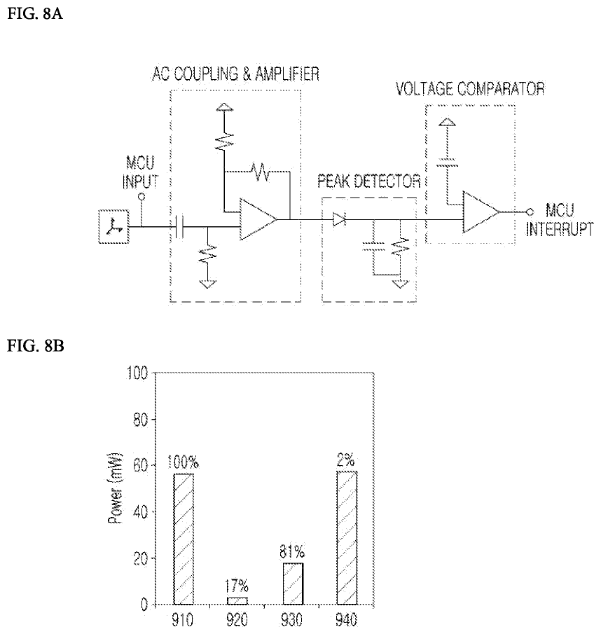

FIG. 8A shows a block diagram illustrating a configuration of a switch circuit included in a wearable device 100 according to an embodiment, and FIG. 8B shows a diagram illustrating a comparison result of power consumption according to the presence or absence of the switch circuit.

Referring to FIG. 8A, the wearable device 100 may further include a switch circuit 140. In an embodiment, the switch circuit 140 may be electrically connected to the acceleration sensor 110 and the processor 120 and may be configured to supply power to preset main components (e.g., a part of hardware) of the wearable device 100 only when acceleration greater than or equal to a preset size is measured through the acceleration sensor 110. To this end, in an embodiment, the switch circuit 140 may be implemented to include an MCU input, an alternating current (AC) coupling and amplifier, a peak detector, a voltage comparator, and an MCU interrupt.

Referring to FIG. 8B, it can be seen that the wearable device 100 including the switch circuit 140 for low power implementation has a total power consumption that is more than 20 times smaller than a case in which the wearable device 100 does not include the switch circuit 140.

Considering that the time for which a human body moves per day is about 4.45 hours on average, the use of the switch circuit 140 having a low power consumption that operates the entire device only when movement of a specific size or greater occurs in the human body may provide a technical effect of consuming a total power reduced by about 4.4 times.

In addition, assuming that an epileptic seizure lasts for about five minutes once a day, the actual time required for wireless communication is only about 2% (=5/(4.45×60)×100) of the total MCU operating time. Accordingly, performing wireless communication with the external device 200 only when an epileptic seizure occurs while selectively using two characteristics according to the above-described acceleration processing method may provide a technical effect of reducing the total power consumption by about 8.8 times.

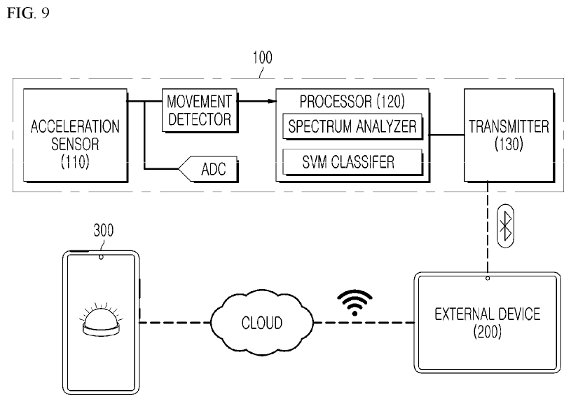

FIG. 9 is a block diagram illustrating a configuration of a wearable healthcare sensor system 1000 according to another embodiment.

Referring to FIG. 9, the wearable healthcare sensor system 1000 may further include a user terminal 300.

The user terminal 300 may correspond to a computer device associated with the wearable device 100 and may be, for example, a device such as a computer used by a guardian of a patient or medical staff in charge to which the wearable device 100 is attached. In an embodiment, the user terminal 300 may be implemented as various types of handheld-based wireless communication devices, such as a mobile phone, a smart phone, a personal digital assistant (PDA), a portable multimedia player (PMP), a tablet personal computer (PC), or the like, or may include various types of wired and wireless communication devices, such as a desktop PC, a tablet PC, and a laptop PC, that have a foundation for installing and executing applications in connection with an external server.

The external device 200 may be connected to one or more wearable devices 100 through a wireless network (e.g., Bluetooth, etc.) to store and manage information received from the wearable device 100. In addition, the external device 200 may be connected to one or more user terminals 300 through a network (e.g., local area network (LAN), Wi-Fi, etc.), and may provide an alarm signal received from the wearable device 100 to a user terminal 300 associated with the wearable device 100. For example, the external device 200 may store and manage a patient identifier of a patient, to which the wearable device 100 is attached, a guardian identifier of a guardian associated with the patient, and a representative identifier of a representative to match a device identifier of the wearable device 100 and, when an alarm signal for a specific disease is received from the wearable device 100, may transmit a warning message indicating that an abnormality for the disease has been detected to the user terminal 300 with the guardian identifier or representative identifier that matches the device identifier of the wearable device 100.

In one embodiment, the processor 120 implemented as an MCU in the wearable device 100 may include a spectrum analyzer that analyzes a power spectrum for acceleration in the above-described manner and an SVM classifier that determines whether an abnormality exists on the basis of the SVM, and the transmitter 130 may include an RF module for wireless transmission and reception and may further include a movement detector for detecting a movement according to the magnitude of the acceleration measured by the acceleration sensor 110, an analog-digital converter (ADC) for signal conversion, the switch circuit 140, and the like.

In addition, the wearable healthcare sensor system 1000 may further include general-purpose components in addition to the components shown in FIG. 1 or FIG. 9. According to other embodiments, some of the components shown in FIG. 1 or FIG. 9 may be omitted.

It should be understood that the order and combination of the operations shown above is for implementing an embodiment, and the order, combination, branch, function and an agent of the operations may be implemented in various forms that are subject to various additions, omissions, or modification within a range without departing from essential characteristics of each component described in the specification. In addition, throughout the specification, an expression “providing” may be interpreted as including a process in which an object acquires specific information or directly or indirectly transmits or receives specific information to or from a specific object, and comprehensively including the performance of relevant operations required in the process.

The various embodiments of the present disclosure may be realized by software including one or more instructions stored in a storage medium (e.g., the memory) that can be read by a machine (e.g., a display apparatus or computer). For example, a processor (e.g., the processor) of the machine may invoke and execute at least one instruction among the stored one or more instructions from the storage medium. Accordingly, the machine operates to perform at least one function in accordance with the invoked at least one instruction. The one or more instructions may include codes generated by a compiler or codes executable by an interpreter. The machine-readable storage medium may be provided in the form of a non-transitory storage medium. Here, when a storage medium is referred to as “non-transitory,” it can be understood that the storage medium is tangible and does not include a signal (for example, electromagnetic waves), but does not distinguish between data being semi-permanently or temporarily stored in the storage medium.

According to one embodiment, the methods according to the various embodiments disclosed herein may be provided in a computer program product. The computer program product may be traded between a seller and a buyer as a product. The computer program product may be distributed in the form of a machine-readable storage medium (e.g., compact disc-read only memory (CD-ROM)) or may be distributed directly between two user devices (e.g., smartphones) through an application store (e.g., Play Store™), or online (e.g., downloaded or uploaded). In the case of online distribution, at least a portion of the computer program product may be stored at least semi-permanently or may be temporarily generated in a machine-readable storage medium, such as a memory of a server of a manufacturer, a server of an application store, or a relay server.

Those skilled in the art should appreciate that various modifications, changes, and substitutions thereto are possible without departing from the scope and spirit of the disclosure. Therefore, the methods disclosed above should be construed as being illustrative rather than limiting the present disclosure. The scope of the disclosure is set forth in the following claims rather than the above specification, and it is intended that the present disclosure covers all modifications provided they come within the scope of the appended claims and their equivalents.

### DESCRIPTION OF REFERENCE NUMERALS

1000: wearable healthcare sensor system
100: wearable device
110: acceleration sensor
120: processor
130: transmitter
200: external device

1. A method of processing acceleration information by a wearable device, the method comprising:
acquiring the acceleration information indicating acceleration according to movement of the wearable device;
acquiring a plurality of acceleration characteristics including a plurality of characteristics of a power spectrum of the acceleration and a range of the acceleration on the basis of the acceleration information;
determining characteristic values according to two characteristics determined according to a type of a disease to be monitored among the plurality of acceleration characteristics; and
providing an alarm signal in response to the characteristic value falling within an abnormality range.

2. The method of claim 1, wherein the providing of the alarm signal includes, in response to the range of the acceleration exceeding a preset value, transmitting the alarm signal to an external device.

3. The method of claim 1, wherein the plurality of acceleration characteristics includes at least one of:
a first characteristic indicating a range of the acceleration in a time domain;
a second characteristic indicating a root mean square of the acceleration in the time domain; and
a third characteristic indicating an average of the acceleration in the time domain.

4. The method of claim 3, wherein the plurality of acceleration characteristics further include at least one of:
a fourth characteristic indicating a mean frequency for the power spectrum of the acceleration;
a fifth characteristic indicating a frequency including a preset first proportion of a total power for the power spectrum of the acceleration;
a sixth characteristic indicating a frequency including a second proportion of the total power;
a seventh characteristic indicating the total power; and
an eighth characteristic indicating a power ratio at a frequency less than a preset motion noise frequency to the total power.

5. The method of claim 4, wherein, in response to the type of the disease being a first disease indicating a cough, the two characteristics corresponding to the first disease include the fifth characteristic and the eighth characteristic,
in response to the type of the disease being a second disease indicating an epileptic seizure, the two characteristics corresponding to the second disease include the fourth characteristic and the eighth characteristic, and
in response to the type of the disease being a third disease indicating a fall, the two characteristics corresponding to the third disease include the first characteristic and the fourth characteristic.

6. The method of claim 1, wherein the providing of the alarm signal in response to the characteristic value being included in the abnormality range includes determining the abnormality range in one or more ranges using a straight line defined by one or more preset linear equations or a curve defined by a polynomial equation on a two-dimensional (2D) plane.

7. The method of claim 1, wherein the determining of the characteristic values according to the two characteristics includes determining the two characteristics and the abnormality range on the basis of learning data that is previously generated,
wherein the learning data is acquired on the basis of at least one of a support vector classifier (SVC), a decision tree, Naïve Bayes, a random forest, and a neural network.

8. The method of claim 1, wherein the providing of the alarm signal in response to the characteristic value falling within the abnormality range includes determining whether the characteristic value falls within the abnormality range using a hyperplane for the plurality of acceleration characteristics acquired through a support vector machine.

9. A wearable device for processing acceleration information, the wearable device comprising:
an acceleration sensor configured to acquire the acceleration information which indicates acceleration according to movement of the wearable device;
a processor configured to acquire a plurality of acceleration characteristics including a plurality of characteristics of a power spectrum of the acceleration and a range of the acceleration based on the acceleration information and determine characteristic values according to two characteristics determined according to a type of a disease to be monitored among the plurality of acceleration characteristics; and
a transmitter configured to provide an alarm signal in response to the characteristic value falling within an abnormality range.

10. The wearable device of claim 9, wherein the transmitter is configured to, in response to the range of the acceleration exceeding a preset value, transmit the alarm signal to an external device.

11. The wearable device of claim 9, wherein the plurality of acceleration characteristics includes at least one of:
a first characteristic indicating a range of the acceleration in a time domain;
a second characteristic indicating a root mean square of the acceleration in the time domain; and
a third characteristic indicating an average of the acceleration in the time domain.

12. The wearable device of claim 11, wherein the plurality of acceleration characteristics further include at least one of:
a fourth characteristic indicating a mean frequency for the power spectrum of the acceleration;
a fifth characteristic indicating a frequency including a preset first proportion of a total power for the power spectrum of the acceleration;
a sixth characteristic indicating a frequency including a second proportion of the total power;
a seventh characteristic indicating the total power; and
an eighth characteristic indicating a power ratio at a frequency less than a preset motion noise frequency to the total power.

13. The wearable device of claim 12, wherein, in response to the type of the disease being a first disease indicating a cough, the two characteristics corresponding to the first disease include the fifth characteristic and the eighth characteristic,
in response to the type of the disease being a second disease indicating an epileptic seizure, the two characteristics corresponding to the second disease include the fourth characteristic and the eighth characteristic, and
in response to the type of the disease being a third disease indicating a fall, the two characteristics corresponding to the third disease include the first characteristic and the fourth characteristic.

14. The wearable device of claim 9, wherein the processor is configured to determine the abnormality range in one or more using a straight line defined by one or more preset linear equations or a curve defined by a polynomial equation on a two-dimensional (2D) plane.

15. The wearable device of claim 9, wherein the processor is configured to determine the two characteristics and the abnormality range on the basis of learning data that is previously generated,
wherein the learning data is acquired on the basis of at least one of a support vector classifier (SVC), a decision tree, Naive Bayes, a random forest, and a neural network.

16. A recording medium on which a program for executing the method of claim 1 is recorded.

17. A recording medium on which a program for executing the method of claim 2 is recorded.

18. A recording medium on which a program for executing the method of claim 3 is recorded.

19. A recording medium on which a program for executing the method of claim 4 is recorded.

20. A recording medium on which a program for executing the method of claim 5 is recorded.
